(19) United States
(12) Patent Application Publication
(10) Pub. No.: US 2020/0320239 A1
(43) Pub. Date: Oct. 8, 2020

(54) METHOD OF EXPLOITING A FRACTURED OIL RESERVOIR HAVING A HETEROGENEOUS PORE SIZE

(71) Applicant: IFP Energies nouvelles, Rueil-Malmaison Cedex (FR)

(72) Inventors: Carlos NIETO DRAGHI, RUEIL-MALMAISON CEDEX (FR); Didier Yu DING, RUEIL-MALMAISON CEDEX (FR); Nicolas SOBECKI, RUEIL-MALMAISON CEDEX (FR); Yu Shu WU, RUEIL-MALMAISON CEDEX (FR)

(21) Appl. No.: 16/841,160

(22) Filed: Apr. 6, 2020

(30) Foreign Application Priority Data
Apr. 4, 2019 (FR) 19/03.635

(51) Int. Cl. G06F 30/28; G01N 33/24; G01N 15/10; G01N 15/08

(52) U.S. Cl. CPC G06F 30/28; G01N 33/241; G06F 2113/08; G01N 15/0886; G01N 15/1012

(57) ABSTRACT

The invention simulates flows in a geological reservoir having a heterogeneous pore size. From laboratory measurements on samples taken in the geological reservoir, pore size distribution classes are determined and a triple-porosity model representative of each class is determined. The flow simulator according to the invention implements the triple-porosity model, a thermodynamic equation of state accounting for an equivalent dimension of the pores of the small-size medium, fluid exchanges exclusively between the large-pore and small-pore media and between the small-pore and fracture media, and the capillary pressure as a function of the saturation in a small-pore medium.

### CROSS-REFERENCE TO RELATED APPLICATIONS

This application claims priority from French Patent application No. 19/03,635, filed Apr. 4, 2019, the contents of which are incorporated herein by reference in their entirety.

### BACKGROUND OF THE INVENTION

### Field of the Invention

The present invention relates to the field of exploration and exploitation of oil reservoirs or of geological gas storage sites. More particularly, the present invention relates to the exploitation of unconventional hydrocarbon reservoirs of very low permeability known as at least one of shale gas plays and tight gas plays.

### Description of the Prior Art

In general, exploration and exploitation of geological oil reservoirs requires knowledge of the underground geology as precise as possible so as to efficiently provide reserves evaluation, production modelling or exploitation management. Indeed, determining the location of at least one of a production well and of an injection well within a hydrocarbon reservoir, the drilling mud composition, the completion characteristics, selection of a hydrocarbon recovery method (such as waterflooding for example) and of the parameters required for implementing this method (such as injection pressure, production flow rate, etc.) requires knowledge of the reservoir. Reservoir knowledge notably is as accurate a description as possible of the structure, the petrophysical properties, the fluid properties, etc., of the reservoir being studied.

To acquire such knowledge, the petroleum industry combines field measurements (performed in situ, during seismic surveys, measurements in wells, core drilling, etc.) with experimental modelling (performed in the laboratory) and numerical simulations (using softwares). Formalization of this knowledge then involves establishing a model of the subsoil, referred to as geological model, which accounts for these aspects in an approximate manner. Generally, this type of model is represented on a computer, and is referred to as numerical model. A geological model generally has a grid cell size of the order of ten meters.

In order to reproduce or to predict (that is “simulate”) hydrocarbon production, reservoir engineering specialists use a computer-implemented flow simulation software also referred to as reservoir simulator. The reservoir simulator calculates the flows and the evolution of the pressures within the reservoir represented by a reservoir model. If the computing power available for the flow simulations permits, the reservoir model can merge with the geological model. In the opposite case, the reservoir model can be obtained by use of an upscaling technique allowing conversing the geological model (model with finer cells) in to the reservoir model (model with coarser cells). This upscaling stage is well known to reservoir engineering specialists and it can be carried out for example using the CobraFlow™ software (IFP Energies nouvelles, France). The cell size of a reservoir model is generally of the order of one hundred meters.

Thus, in general terms, a flow simulator calculates the spatial and temporal evolution of the flow and the thermodynamics of fluids contained in a reservoir, as well as the production in production wells located in this reservoir.

As the global oil demand increases, the development of low-permeability unconventional oil plays has attracted the interest of the petroleum industry for several years as an alternative to the conventional oil reservoirs exploited so far.

Now, the flows and thermodynamics of hydrocarbon fluids in a low-permeability unconventional play are much more complex than in conventional plays in several aspects. First, the permeability of the porous matrix is very low, which is on the order of several ten nano-Darcies. The porous matrix therefore needs to be stimulated by multistage hydraulic fracturing in order to generate a very heterogeneous fractured porous medium, where the flow occurs between a very low permeability porous matrix and the fractures to the wells. The very low permeability of the matrix involves a very slow matrix/fracture transient flow regime. Furthermore, unlike the usual pore size of a conventional reservoir of the order of one micrometer, the pore size of very low permeability reservoirs is of the order of one nanometer. The size of the molecules of a hydrocarbon ranging between 0.5 and 10 nm, the molecular interaction forces of Van der Waals type between the fluid and the wall of a nanometric pore become as significant as the interaction forces between fluid molecules. The thermodynamic behavior of the fluid is thus greatly modified, the PVT (pressure-volume-temperature) properties of the fluid at liquid/vapour equilibrium and the bubble point differ greatly from a case where the fluid is not confined and they become dependent of the pore size. Finally, the pore size distribution in a very low permeability oil play is very heterogeneous, from one nanometer to one millimeter, which causes a very significant spatial heterogeneity of the capillary pressure and the thermodynamic behaviour within the porous matrix.

The following documents are mentioned in the description hereafter:
Alfi, M., An, C., Cao, Y. et al. 2017. Pore Size Variability and Sieving Effect in Liquid Shale-A Multiple Permeability Approach and Eagle Ford Case Study. SPE Reservoir Simulation Conference, 20-22 February, Montgomery, Tex., USA. https://doi.org/10.2118/182643-MS.
E. P. Barret, L. G. Joyner, P. B. Halenda J. Am. Chem. Soc., 73 (1951), p. 373. Bird, Robert Byron; Stewart, Warren E.; Lightfoot, Edwin N. (2007) Transport phenomena. Revised 2nd edition. New York etc.: John Wiley & Sons Inc (1).
Chalmers, Gareth R.; Bustin, R. Marc; Power, Ian M. (2012) Characterization of Gas Shale Pore Systems by Porosimetry, Pycnometry, Surface Area, and Field Emission Scanning Electron Microscopy/Transmission Electron Microscopy Image Analyses. Examples from the Barnett, Woodford, Haynesville, Marcellus, and Doig units. In: AAPG Bulletin, vol. 96, no 6, p. 1099-1119. DOI: 10.1306/10171111052.
Jin, Luchao; Ma, Yixin; Jamili, Ahmad (2013) Investigating The Effect of Pore Proximity on Phase Behavior And Fluid Properties in Shale Formations. SPE Annual Technical Conference and Exhibition. Richardson, Tex.
Kuila, Utpalendu; Prasad, Manika (2011) Understanding Pore-Structure And Permeability In Shales: SPE Annual Technical Conference and Exhibition, 30 October-2 November, Denver, Colo., USA. https://doi.org/10.2118/146869-MS
Kuila, Utpalendu; Prasad, Manika (2013) Specific surface area and pore-size distribution in clays and shales. In: Geophysical Prospecting, vol. 61, no 2, p. 341-362. DOI: 10.1111/1365-2478.12028.
Le Ravalec M, Noetinger B, Hu L Y (2002) The FFT Moving Average (FFT-MA) Generator: An Efficient Tool for Generating and Conditioning Gaussian Simulations. Math Geol 32(6):701-723.
Meyra, Ariel G.; Zarragoicoechea, Guillermo J.; Kuz, Victor A. (2005) Thermodynamic Equations For a Confined Fluid at Nanometric Scale. In: Fluid Phase Equilibria, vol. 230, no 1-2, p. 9-14. DOI: 10.1016/j.fluid.2004.10.014.
Pruess, K. (1985). A Practical Method for Modeling Fluid and Heat Flow in Fractured Porous Media. Society of Petroleum Engineers. doi:10.2118/10509-PA.
Warren, J. E. et Root, P. J., “The Behavior of Naturally Fractured Reservoirs”, SPE Journal (September 1963), 245-255.

“Dual medium” or “dual porosity” type approaches are conventionally used to represent the complexity of fractured media. This approach, described for example in (Warren and Root, 1963), involves that any elementary volume (reservoir model cell) of the fractured reservoir is modelled in form of a set of identical parallelepipedic blocks, referred to as matrix blocks, delimited by an orthogonal system of continuous uniform fractures oriented in the principal directions of flow. The fluid flow, at reservoir scale, essentially occurs through the fractures, and fluid exchanges occur locally between the matrix blocks and the fractures. Most often, the cells have hectometric lateral dimensions (commonly 100 or 200 m) considering the size of the fields and the limited possibilities of simulation softwares in terms of computing capacity and time. The result thereof is that, for most fractured fields, the fractured reservoir elementary volume (cell) comprises innumerable fractures forming a complex network that delimits multiple matrix blocks of variable dimensions and shapes according to the geological context. It is then possible to formulate and to calculate the matrix-fracture exchange flows for this representative matrix block and to multiply the result by the number of such blocks in the elementary volume (cell) to obtain the flow at the scale of this cell.

This type of dual-porosity representation is however not suitable in the case of very low permeability reservoirs subjected to hydraulic fracturing stimulation for example. Indeed, such a model does not account for the very long transient regime of the matrix/fracture flow due to the very low permeability of the matrix. Furthermore, this type of model does not take the fluid thermodynamics depending on the pore size into account.

Furthermore, the document (Alfi et al., 2017) discloses a model referred to as “triple porosity”, where the fractured medium is divided into a medium representative of the fractures, a medium representative of the larger pores of the matrix medium and a medium representative of the smaller pores of the matrix medium. In other words, in relation to the dual-porosity model, the triple-porosity model according to (Alfi et al., 2017) divides the matrix medium into two sub-media, a medium representative of the porous matrix pores of larger size and a medium representative of the porous matrix pores of smaller size. Besides, the model described in this document implements a conventional thermodynamic equation of state, such as the Peng-Robinson equation of state for the “large-pore” medium and the “fracture” medium, and a thermodynamic equation of state as described in (Travalloni et al., 2014) for the “small-pore”medium. The thermodynamic equilibrium between small pores and large pores is verified.

The flow is possible between all the media and no discretization of the matrix medium is performed. The very slow transient regime between matrix and fracture is therefore not taken into account. Furthermore, no verification of the model by means of a fine-scale simulation is carried out.

### SUMMARY OF THE INVENTION

Thus, the present invention improves flow simulation in the case of a geological reservoir characterized by a heterogeneous pore size distribution, ranging from one nanometer to one millimeter, with a view to a better prediction of the production of fluid contained in this reservoir. More precisely, the present invention relates to a method for simulating the flows of a fluid contained in a geological reservoir having a high pore size heterogeneity, flow simulation being performed by use of a flow simulator implementing at least one triple-porosity model, and allowing accounting for the very slow transient regime between porous matrix and fractures, the thermodynamics depending on the pore size and the capillary pressure heterogeneities.

The invention further relates to a method of exploiting a fluid contained in a fractured geological reservoir characterized by a heterogeneous pore size distribution, using a flow simulation method as described below in order to predict production curves for this fluid over time as a function of various exploitation schemes for this reservoir, and thus to determine an optimum exploitation scheme for producing this fluid.

The present invention relates to a computer-implemented method for simulating fluid flows, the fluid comprising an oil phase and a gas phase, in a fractured geological reservoir having a heterogeneous pore size distribution, wherein a first gridded representation of the reservoir is constructed from the measurement of properties relative to the reservoir. The method according to the invention comprises at least the following steps:
A. from measurements performed in the laboratory on a plurality of samples from various spatial positions in the geological reservoir, determining pore size distribution classes and assigning a pore size distribution class to each of the cells of the first gridded representation;
B. for each of the pore size distribution classes, constructing a porosity model representative of the pore size distribution, the porosity model comprising a first medium representative of the pores of the distribution whose dimension is in a first range, a second medium representative of the pores of the distribution whose dimension is in a second range and a third medium representative of the fractures of the distribution, the dimensions of the pores of the first range being greater than the dimensions of the pores of the second range, the porosity model being further described by flow parameters for each of the media, the flow parameters comprising at least one equivalent dimension of the pores of the second medium and a capillary pressure depending on a saturation with the gas phase of the fluid in the second medium,
C. for each of the pore size distribution classes, calibrating at least part of the parameters of the porosity model representative of the pore size distribution and assigning the calibrated porosity model in each of the cells of the first gridded representation to which the class is assigned.

Then, according to the invention, the flows of the fluid in the geological reservoir are simulated by means of the first gridded representation and of a first flow simulator, the first simulator implementing at least:
the calibrated porosity models assigned in each of the cells of the first gridded representation,
for the second medium, a thermodynamic equation of state taking account of the equivalent dimension of the pores of the second medium,
within the porosity models, exchanges of the fluid between the first medium and the second medium and between the second medium and the third medium of a single porosity model, by taking account of the capillary pressure depending on saturation with the gas phase of the fluid in the second medium,
between at least two of the porosity models, exchanges of the fluid exclusively between the third media of the two porosity models.

According to an implementation of the invention, the calibration for one of the pore size distribution classes can be performed with the following steps:

a) simulating the flows by use of the first simulator and of the porosity model representative of the pore size distribution class, the porosity model being defined by the flow parameters of the model, and obtaining production curves depending on the flow parameters of the porosity model;
b) measuring the differences between the production curves depending on the flow parameters of the porosity model and predetermined reference production curves, and correcting at least part of the flow parameters of the porosity model so as to minimize the differences;
c) reiterating steps a) and b) until the differences are below a predetermined threshold, the flow parameters of the porosity model for an iteration of the reiteration corresponding to the corrected parameters in a previous iteration of the reiteration.

According to an implementation of the invention, the reference curves can be determined with the following steps:

i) constructing a second gridded representation representative of the pore size heterogeneity of the pore size distribution class, the dimensions of the cells of the second gridded representation being determined so as to account for the effects induced by the pore size of the distribution;
ii) simulating flows by using the second gridded representation and of a second flow simulator implementing at least one thermodynamic equation of state taking accounting for the dimensions of the pores of the pore size distribution class, and obtaining production curves of the fluid relative to the second gridded representation.

According to an implementation of the invention, the part of the corrected flow parameters can comprise the equivalent dimension of the pores of the second medium, an equivalent permeability and porosity of the second medium or an equivalent permeability and porosity of the first medium, and a parameter representative of the proportionality of the transmissibility from the first medium to the second medium.

Advantageously, the laboratory measurements can be performed by using a mercury porosimetry method and a nitrogen adsorption/desorption method.

According to an implementation of the invention, the first medium and second medium can be discretized using a nested ring type discretization.

Furthermore, the invention relates to a computer program product which may be downloaded from a communication network and obtained from a computer-readable medium or executed by a processor executing programing code instructions for implementing the method as described above, when the program is on a computer.

The invention also relates to a method of exploiting a fluid contained in a fractured geological reservoir having a heterogeneous pore size distribution, wherein the computer-implemented method is applied to simulate flows of a fluid as described above, and wherein, from at least the simulation of the flows in the geological reservoir, an exploitation scheme comprising at least one site for at least one injection well and at least one production well which is determined for the geological reservoir, and the fluid of the geological reservoir is exploited at least by drilling the wells of the site and by providing them with exploitation infrastructures.

### BRIEF DESCRIPTION OF THE DRAWINGS

Other features and advantages of the method according to the invention will be clear from reading the description hereafter of embodiments given by way of non limitative example, with reference to the accompanying figures wherein:

FIGS. 1a and 1b illustrate a section along a horizontal plane in a distribution of the equivalent permeabilities of the fracture medium of a geological reservoir and in a distribution of 5 pore size distribution classes of the matrix medium of this geological reservoir;

FIG. 2 illustrates a section along a horizontal plane in a fine-scale gridded representation of a pore size distribution;

FIGS. 3a to 3c compare reference production curves with production curves obtained for a pore distribution class example, after calibration according to step 3 of the method according to the invention,

FIGS. 4a to 4c compare production curves obtained with the method according to the invention, with a method according to the prior art and with in-situ measurements, and

FIGS. 5a to 5i illustrate oil pressure fields, gas pressure fields and gas saturation fields for each of the media of the triple-porosity model according to the invention.

### DETAILED DESCRIPTION OF THE INVENTION

In general terms, the first aspect of the invention relates to a method for simulating fluid flows in a fractured geological reservoir having a heterogeneous pore size. A heterogeneous pore size is understood to be a pore size generally ranging from one nanometer to one millimeter. The fluid contained in the geological reservoir study comprises at least an oil phase and a gas phase.

According to a second aspect, the invention relates to a method for exploiting a fluid contained in a fractured geological reservoir having a heterogeneous pore size, by use of a method for simulating fluid flows in a geological reservoir according to the first aspect of the invention.

The present invention requires:
property measurements relative to the geological reservoir being studied which are, on the one hand, petrophysical property measurements performed in situ or in the laboratory, such as porosity, permeability, lithology (i.e. the rock type), relative permeability or capillary pressure. These measurements may have been obtained for example through laboratory analysis of core samples taken in situ, through logging operations carried out in wells traversing the reservoir being studied, or through seismic acquisition surveys. On the other hand, these measurements relate to the properties of the fluids flowing through the geological reservoir studied, such as oil flow rate, water flow rate, pressure or saturation measurements. These measurements may have been obtained for example by producing the fluid in some wells traversing the geological reservoir being studied, during well tests or interference tests. Generally, such property measurements are well known in the field of flow simulation in a geological reservoir;
a gridded representation representative of at least the geological reservoir being studied: also referred to as reservoir model which is a subsoil model constructed in order to describe as precisely as possible the structure, the petrophysical properties and the properties of the fluids in the reservoir being studied. This model is generally represented on a computer and it has a grid, each cell of this grid comprising one or more property values relative to the reservoir being studied (such as porosity, permeability, saturation, geological facies, pressure, etc.). A reservoir model must verify as far as possible the properties collected in the field: logging data measured along wells, measurements performed on rock samples obtained via core drilling for example, data deduced from seismic acquisition surveys, production data such as oil flow rate, water flow rate, pressure variations, etc. Reservoir simulation can be performed with the methods for constructing such a gridded representation of a geological reservoir. It is noted that the reservoir model can merge with the geological model when the computing power is sufficient to allow numerical flow simulation calculations on a fine-cell grid. Otherwise, an upscaling technique can be used to convert a fine-cell model (geological model) to a model with coarser cells (reservoir model). This upscaling step can be carried out for example with the CobraFlow™ software (IFP Energies nouvelles, France). According to an implementation of the invention, the gridded representation of the geological reservoir being studied comprises at least a saturation value, a pressure value, a temperature value, a composition of the fluid mixture, a permeability, a porosity, a capillary pressure, a relative permeability and a pore size in each of the cells thereof;
a flow simulator according to the invention: a flow simulator is a numerical program executed on a computer, which is used to simulate fluid flows, in the present case, in a geological reservoir. Flow simulation, also referred to as reservoir simulation in this case, consists in numerically predicting the production over time of a fluid trapped in a geological reservoir, production requiring the existence of at least one production well (towards which the trapped fluid is displaced by pressure gradient and from where it can be extracted). The flow simulator according to the invention, which notably implements a triple-porosity model, is described in detail in step 3 hereafter. According to an implementation of the invention, the simulator according to the invention can further implement a single-porosity model, as described in step 4 hereafter.

The method according to the first aspect of the invention comprises at least steps 1 to 4 described hereafter. The method according to the second aspect of the invention comprises at least steps 1 to 5 described hereafter.

1. Determining Pore Size Distribution Classes

According to the invention, laboratory measurements are performed on rock samples taken in various spatial positions (for example represented by positions (x,y,z) in a geographic reference frame) of the geological reservoir being studied, and at least one pore size distribution relative to each of the samples is determined from these measurements. Then, according to the invention, from at least these measurements, pore size distribution classes are determined and a pore size distribution class is assigned to each sample. In other words, the various pore size distributions thus measured are grouped by category or by type. For example, if pore size distribution measurements are performed on 50 distinct samples, resemblances can be determined between these 50 pore size distributions and they can be organized into 5 classes for examples. According to an implementation of the invention, at least three pore size distribution classes are preferably determined. A class can be identified by a number, a letter, a combination of numbers, a combination of letters or any other identifier.

According to an implementation of the invention, this classification can take account of the porosity and the permeability associated with each of the samples. It may be the porosity and the permeability measured directly on each of the samples, or the porosity and the permeability in the cell of the gridded representation of the geological reservoir to which this sample belongs. Indeed, there is a direct connection between porosity and permeability on the one hand and pore size distribution on the other hand, so that a classification applied to porosity and permeability can be advantageous for determining pore size distribution classes.

According to a preferred implementation of the invention, the laboratory measurements for determining the pore size distribution of a rock sample from the geological reservoir studied comprise the combination of the mercury intrusion porosimetry (MIP) method and of the nitrogen gas adsorption/desorption (N2GA) method. The mercury porosimetry method is a conventional method for determining a pore size distribution. Mercury is a non-wetting liquid that does not penetrate the pores spontaneously by capillarity, the application of pressure is required. The mercury penetrates the pores under the effect of an increasing pressure in increasingly smaller pores. Measurement of the intrusion volume as a function of pressure allows determination the pore size distribution. In particular, the Washburn equation allows linking pressure and pore size. However, the mercury porosimetry method is not capable of taking the pores of very small size into account. A nitrogen (N2) adsorption/desorption method is therefore advantageously implemented as a complement to the MIP method. The pore size distribution can be obtained from the adsorption and desorption isotherms. The most commonly used model is the one described in (Barret et al., 1951). Thus, the combination of measurements using the MIP and N2GA methods is advantageous because, insofar as the MIP method does not allow describing the complete structure of the smaller pores, it is complemented by the N2GA method, which is notably capable of measuring pores of a diameter below 200 nm.

According to another implementation of the invention, other suitable methods may also be used to determine a rock sample pore size distribution, as described in the documents (Kuila and Prasad, 2011; Kuila and Prasad, 2013; Chalmers et al., 2012).

According to an implementation of the invention, a classification of the plurality of pore size distributions is carried out using the classification method known as K-means. Generally speaking, the K-means algorithm allows grouping the values of variables into K classes that do not overlap. A number of classes (or coefficient K) generally below 10 is selected in order to obtain a relatively stable result. This algorithm is advantageously of simple design, high timeliness and low memory requirements.

According to an implementation of the invention, after determining pore size distribution classes from the plurality of laboratory measurements performed on the plurality of rock samples from the geological reservoir being studied, a pore size distribution class being then assigned to each of the samples, a class is assigned in each cell of the gridded representation. According to an implementation of the invention, from the spatial position, in the geological reservoir studied, of each of the samples, the cell of the gridded representation to which they belong is deduced, the pore size distribution class determined for each of these samples is assigned in respective cells thus identified, then, for example by use of a geostatistical method as described in the document (Le Ravalec et al., 2002), a pore size distribution class is assigned in each cell of the gridded representation for which no pore size distribution class has been assigned. This may be the case for cells where no rock sample has been taken and therefore cells for which no direct pore size distribution measurement was available.

2. Constructing a Triple-Porosity Model for Each Pore Size Distribution Class

The second step of the method according to the invention is applied for each one of the pore size distribution classes identified at the end of step 1 described above.

According to the invention, for each pore size distribution class determined in step 1, a model known as triple-porosity model, representative of the pore size distribution class which is considered, is constructed. More precisely, the triple-porosity model according to the invention is defined as follows: a first medium representative of the pores of the distribution whose dimension is in a first range, a second medium representative of the pores of the distribution whose dimension is in a second range and a third medium representative of the fractures, the dimensions of the pores of the first range being greater than the dimensions of the pores of the second range. In the rest of the description, for simplicity purposes, the first medium is referred to as “large-pore medium”, the second medium is referred to as “small-pore medium” and the third medium is referred to as “fracture medium”. In other words, in a simplified way, two ranges are determined for the pore sizes of each pore size distribution determined in step 1: a first range where the pores are of larger size than those of the second range. According to an implementation of the invention, the first range of the triple-porosity model defines pores having a diameter for example greater than or equal to 100 nm, the second range of the triple-porosity model according to the invention defines pores having a diameter less than 100 nm. Compared to a dual-medium type model, for which a parallelepipedic block representative of the porous matrix is delimited by an orthogonal and regular network of fractures, the triple-porosity model according to the invention represents the porous matrix by two matrix blocks, one representative of the small pores of this matrix and the other representative of the large pores of this matrix.

According to the invention, flow parameters are further assigned for each of the defined three media of the triple-porosity model. According to the invention, at least one equivalent (or effective) dimension (the radius for example) of the pores of the small-pore medium is assigned. Besides, according to the invention, a capillary pressure depending on the gas saturation is assigned to the small-pore medium only, and a zero capillary pressure is assigned to the large-pore medium.

Implicitly, the flow parameters of the porosity model further comprise an equivalent (or effective) porosity and permeability for each medium making up the porosity model, as well as a relative permeability and a capillary pressure for each medium making up the porosity model.

According to an implementation of the invention, the flow parameters of the porosity model comprise the equivalent dimension of the pores of the small-pore medium, the equivalent permeability and porosity of the small-pore medium (or, in an equivalent manner, the equivalent permeability and porosity of the large-pore medium), and a parameter α representative of the proportionality of the transmissibility from the large-pore medium to the small-pore medium. According to this implementation, the sum of the small-pore and large-pore permeabilities (respectively porosities) is equal to the permeability determined for the pore size distribution class considered. As described in step 3, the initial distribution between the small-pore and large-pore permeability (respectively porosity) distribution can be updated using a calibration method.

According to an implementation of the invention, the method described in patent application EP-3,181,804 corresponding to US published application 2017-0,212,276) can be used to determine the size of the matrix blocks. The FracaFlow® software (IFP Energies nouvelles, France) can generally be used to determine equivalent fracture permeabilities and porosities.

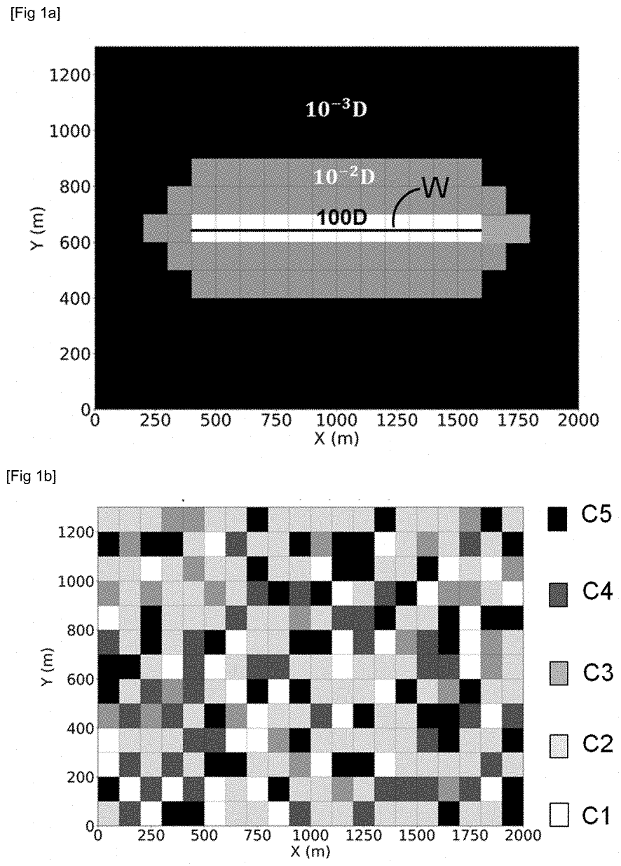

By way of example, FIGS. 1a and 1b illustrate a section at constant z (or a horizontal plane) in a gridded representation of a geological reservoir showing the distribution of the equivalent permeabilities in the fracture medium (FIG. 1a) and the distribution of 5 pore size distribution classes denoted by C1 to C5 (each identified by a specific colour, FIG. 1b). A very high permeability (100 D) is observed in the fracture medium along horizontal segment W representative of a well, and it steeply decreases (from 10⁻² D to 10⁻³ D) as the distance to this well increases. The well is intended for hydraulic stimulation of the fractured medium, which explains the very high permeability of the fractures close to the fracturing well.

3. Calibrating at Least Part of the Flow Parameters of the Porosity Models Determined for Each of the Pore Size Distribution Classes

The third step of the method according to the invention is applied for each pore size distribution class identified in step 1 above and for which a porosity model has been constructed as described in step 2 above.

According to the invention, for each pore size distribution class, at least part of the flow parameters of the porosity model constructed for this pore size distribution class is to be calibrated.

According to an implementation of the invention, for a given pore size distribution class, the flow parameters of this class are calibrated using predetermined reference production curves, and by carrying out the following steps:

a) simulating flows by use of the flow simulator according to the invention (see the detailed description of the simulator according to the invention in step 4 below), applied to the porosity model representative of the pore size distribution class as constructed in the previous step, the porosity model being further described by flow parameters. Production curves relative to the porosity model established in step 2, therefore depending on the flow parameters of the porosity model, are obtained,
b) measuring the differences between the production curves relative to the porosity model and the reference production curves, and correcting the parameters of the porosity model to minimize these differences,
c) reiterating steps a) and b) until the differences are below a predetermined threshold, the parameters of the porosity model used for a new iteration of step a) corresponding to the parameters corrected for a previous iteration. According to an implementation of the invention, an optimization algorithm, based for example on the conjugate gradient method, can be used to minimize in an automated manner, and according to an iterative process, an objective function measuring the differences between reference production curves and simulated production curves for flow parameter values at a given iteration.

According to an implementation of the invention, reference production curves are determined by carrying out the following steps:

i) constructing a so-called “fine-scale” gridded representation whose cell dimensions are fine enough to account for the effects induced by the pore size of the distribution considered on the flows. It can generally be considered that such a gridded representation has cells of dimension at least 100 times less than the dimensions of the cells of the gridded representation of the geological reservoir. Thus, the purpose is not to construct a gridded representation representative of the geological reservoir as a whole, but only representative of a portion of the reservoir where matrix/fracture exchanges occur. It is therefore a fine-scale fictitious gridded representation. This gridded representation can for example have cell dimensions of 0.2 m×0.2 m×0.2 m and an overall dimension of 20 m×20 m×20 m,
ii) simulating flows by use of this fine-scale gridded representation and of a flow simulator implementing a single-porosity model and at least one thermodynamic equation of state taking account of the dimensions of the pores of the pore size distribution class. According to an implementation of the invention, the thermodynamic equation of state can enable a critical point modification, or the thermodynamic equation of state corresponds to any other variant. Fluid production curves relative to this fine-scale gridded representation and therefore representative of the fine-scale flows are thus obtained. These production curves can then serve as reference curves for calibrating the flow parameters of the porosity model according to this implementation of the invention.

According to a most preferred implementation of the invention, the flow parameters that are updated in this step are: the equivalent dimension of the pores of the small-pore medium, the equivalent permeability and porosity of the small-pore medium (or, in an equivalent manner, the equivalent permeability and porosity of the large-pore medium) and a parameter α representative of the proportionality of the transmissibility from the large-pore medium to the small-pore medium. The equivalent dimension of the pores of the small-pore medium is characteristic of the pore size distribution and it varies as a function of the fraction of small pores in the porous matrix. The permeability and porosity distribution between small and large pores is also characteristic of the pore size distribution. Parameter α depends on the large pores-small pores contact in the fine simulation.

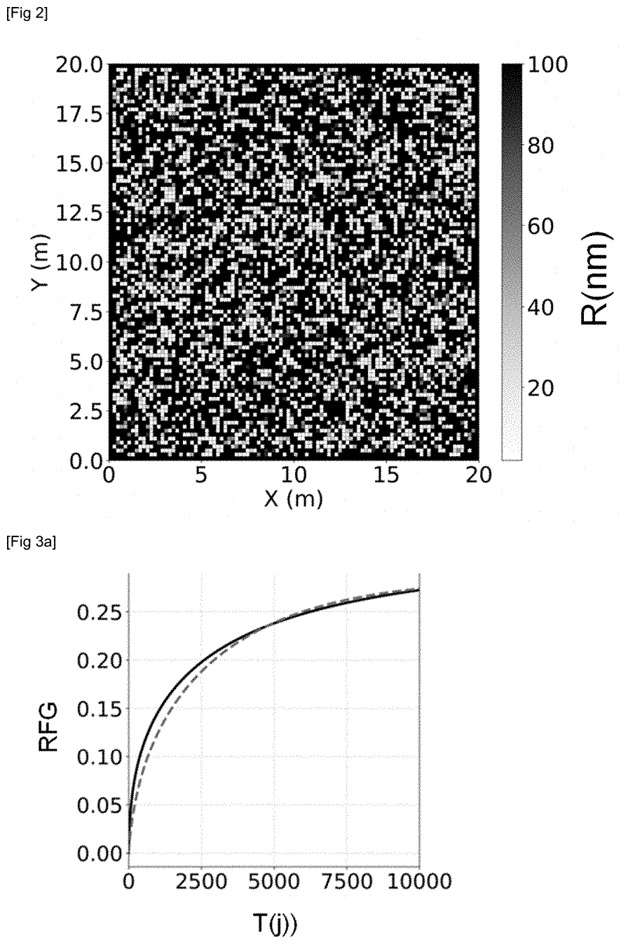

FIG. 2 shows, by way of illustration, a section in a horizontal plane (in a plane at constant z in a geographic reference frame) in a fine-scale gridded representation of a pore size distribution. FIG. 2 shows a high variability of the pore size (radius R) over very short distances (the dimensions of the gridded representation are 20 m in direction X and 20 m in direction Y). A 100 nm value has been chosen here to delimit the small-pore and large-pore media. It can be observed in Table 1 that the pores of larger size (radius above 100 nm) represent more than 80% of the volume fraction of all the pores, while the pores of very small size only represent 20% of the volume fraction of all the pores. The porosity and permeability values of the porous matrix and of the fracture medium are also given in Table 1.

TABLE 1

| | Pore | | | Pore |
|---|---|---|---|---|
| | radius | | | volume |
| Medium | (nm) | Porosity | Permeability | fraction (%) |
| Matrix | 2-100 | 0.036 | 100 nD | 20 |
| | >100 | 0.1 | | 80 |
| Fracture | | 0.001 | 10 D | |

Table 2 gives the flow parameters of the porosity model after calibration according to the most preferred implementation described above. The permeability of the large-pore medium after calibration is 60 nD, which implies that the permeability of the small-pore medium is 40 nD, which amounts to 100 nD for the matrix medium in general. Besides, the inter-large pore flow is higher than the inter-small pore flow because the pores of radius above 100 nm represent 80% of the volume.

TABLE 2

| Equivalent pore radius | Equivalent permeability | |
|---|---|---|
| of the small-pore | of the large-pore | |
| medium (nm) | medium (nD) | Parameter α |
| 6 | 60 | 0.08 |

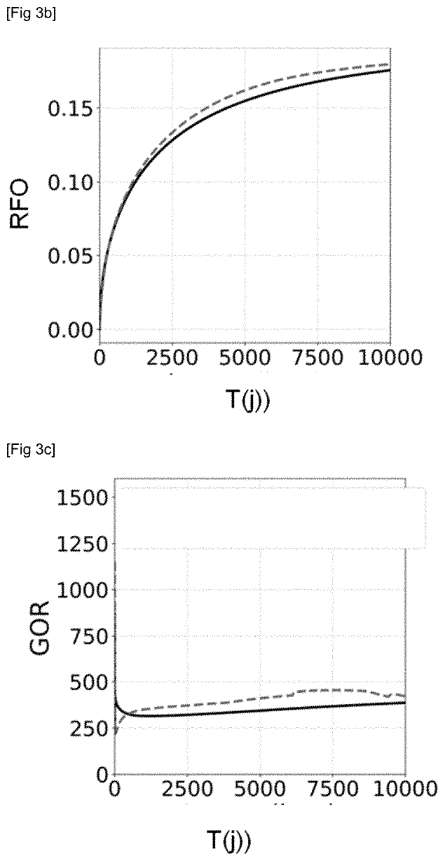

FIGS. 3a, 3b and 3c compare reference production curves (dotted curves) with production curves obtained with the porosity model (solid curves) according to the invention after calibration applied as described in the preferred embodiment above. More specifically, FIGS. 3a, 3b and 3c respectively show the gas recovery factor (RFG), the oil recovery factor (RFO) and the gas-oil ratio (GOR) as a function of time T (in days) obtained with a fine scale (dotted curves) and with the porosity model (solid curves). It is noted in these figures that the production curves obtained on the triple-porosity model according to the invention after calibration are very close to the reference results obtained with the fine-scale simulation. The calibration performed on the three flow parameters consisting of the equivalent pore dimension of the small-pore medium, the equivalent permeability of the small-pore medium (or, in an equivalent manner, the equivalent permeability of the large-pore medium) and parameter α representative of the proportionality of the transmissibility from the large-pore medium to the small-pore medium is thus particularly efficient.

At the end of this step, applied for each of the pore size distribution classes determined at the end of step 1, a calibrated porosity model is obtained for each pore size distribution class.

4. Flow Simulation Using the Porosity Models Determined for Each of the Pore Size Distribution Classes

This step simulates the flows in the geological reservoir by use of a flow simulator according to the invention and of the porosity models determined and calibrated for each pore size distribution class.

According to the invention, a porosity model is first assigned in each cell of the gridded representation. Since at the end of step 1 a pore size distribution class is assigned to each cell of the gridded representation of the geological reservoir, the porosity model determined and calibrated for the class assigned to a given cell just needs to be assigned to this cell.

The flow simulator, also referred to as reservoir simulator, according to the invention allows at least:

a) to account for a triple-porosity model as described in step 2 above. In other words, the flow simulator according to the invention is capable of considering that each cell of the gridded representation is divided into matrix blocks surrounded by fractures, each matrix block being represented by a small-pore medium and a large-pore medium. More precisely, according to the invention, the flow simulator according to the invention implements the calibrated porosity models assigned in each cell of the gridded representation representative of the geological reservoir studied as described in steps 2 and 3 above,
b) to account, for the small-pore medium, of a thermodynamic equation of state taking into account the equivalent dimension of the pores of the small-pore medium. In general, a thermodynamic flash calculation carried out using a thermodynamic equation of state is a calculation of the liquid/vapor equilibrium of a fluid for a given pressure and temperature. The input data of a thermodynamic flash are the molar fractions zᵢ of the mixture components, the pressure and the temperature. The output data are the liquid xᵢ and vapor yᵢ molar fractions of each component i, as well as the liquid and vapour densities. The thermodynamic equation of state according to an embodiment of the invention is based on the Rachford-Rice equation and on the Peng-Robinson equation of state. The Rachford-Rice equation allows, from molar fraction zᵢ and from the equilibrium coefficients of the mixture components Kᵢ, to calculate the vapor molar fraction V with a formula of the type:

$$\sum \text{?} \frac{\text{?} K \text{?} - 1 ) z \text{?}}{1 \vert V ( K \text{?} 1 )} \text{?} 0$$

$$\text{?} \text{indicates text missing or illegible when filed}$$

The Peng-Robinson equation is a conventional analytical expression allowing to relate pressure P, temperature T and molar volume V. It also allows calculation of the fugacity coefficients of each compound in liquid and vapor state.

The liquid/vapour equilibrium state is defined when the fugacity of each component of the mixture in liquid state is equal to the vapor state.

In general, a thermodynamic flash algorithm optimizes the value of equilibrium coefficient Kᵢ until thermodynamic equilibrium and thus equality of the liquid and vapor fugacities of each component.

More precisely, the stages of a thermodynamic flash algorithm are as follows. The initial equilibrium coefficient Kᵢ is calculated from the Wilson analytical solution. The Rachford-Rice equation is solved so as to obtain L and therefore xᵢ and yᵢ, which allow to calculation of the coefficients of the Peng-Robinson cubic equation. A new Kᵢ can thus be calculated from the fugacity coefficients. If the equality of the fugacities is not fulfilled, this new coefficient Kᵢ is used in the Rachford-Rice equation and the algorithm repeats the same stages until equality of the liquid/vapor fugacities of each component is obtained, and thus thermodynamic equilibrium is reached.

According to an implementation of the invention, for the critical pressure, it is possible to use the correlation described in the document (Meyra et al., 2005) for each component, where the distance between particles is such as described in (Bird et al., 2007). According to an implementation of the invention, for the critical temperature, the correlation described in (Jin et al., 2013) can be used.

According to an implementation of the invention, the thermodynamic flash algorithm is unchanged in relation to a thermodynamic flash algorithm of the prior art, but it is the use thereof that is modified, the critical pressure and temperature input values being a function of the pore radius of the small-pore medium.

According to the invention, the pore fraction whose thermodynamic properties and bubble point are modified by the strong fluid/pore interaction in the small-pore medium is thus taken into account,

c) to account for the fluid exchanges occurring as follows exclusively: fluid exchanges from the large-pore medium to the small-pore medium, and fluid exchanges from the small-pore medium to the fracture medium. Thus, such a description of the fluid exchanges excludes fluid exchanges directly between the large-pore medium and the fracture medium, unlike the simulator implementing a triple-porosity model as described in (Alfi et al., 2017). Furthermore, the simulator according to the invention takes account of the capillary pressure as a function of the gas saturation for flow calculation.

According to an implementation of the invention, the flow simulator used for implementing the method according to the invention uses mass conservation equations as described by a system of equations of the type:

$$\{ \begin{matrix}\frac{\partial }{\partial t} [ {ɛ}^{L} {\sum }_{p} {ρ}_{p}^{L} {C}_{kp}^{L} {S}_{p}^{L} ] + div [ {\sum }_{p} {ρ}_{p}^{L} {C}_{kp}^{L} \vec{{u}_{p}^{L}} ] + {F}_{kp}^{LS} = 0 \\ \frac{\partial }{\partial t} [ {ɛ}^{S} {\sum }_{p} {ρ}_{p}^{S} {C}_{kp}^{S} {S}_{p}^{S} ] + div [ {\sum }_{p} {ρ}_{p}^{S} {C}_{kp}^{S} \vec{{u}_{p}^{S}} ] + {F}_{kp}^{Sf} - {F}_{kp}^{LS} = 0 \\ \frac{\partial }{\partial t} [ {ɛ}^{f} {\sum }_{p} {ρ}_{p}^{f} {C}_{kp}^{f} {S}_{p}^{f} ] + div [ {\sum }_{p} {ρ}_{p}^{f} {C}_{kp}^{f} \vec{{u}_{p}^{f}} ] + {\sum }_{p} {ρ}_{p}^{f} {C}_{kp}^{f} {Q}_{p}^{f} - {F}_{kp}^{Sf} = 0\end{matrix}$$

wherein the terms of this system are defined as follows:
exponent L: large pores
exponent S: small pores
exponent f: fracture
p: phase
k: compound of the fluid
ε: porosity
Sₚ: saturation of phase p
Cₖₚ: molar fraction of compound k in phase p
ρₚ: density of phase p
F: flow, with exponent “Sf” meaning from “small pores” to “fractures”, exponent “LS” meaning from “large pores” to “small pores”
u: velocity obtained with Darcy's law.

Such a system describes the accumulation and convective flow terms in each medium, as well as the inter-media exchange flows. According to this implementation of the invention, the well term is disregarded in the small-pore and large-pore media because it is assumed that the main part of the production occurs through the fractures. The inter-media exchange flow terms correspond to the flows from the large pores to the small pores and from the small pores to the fractures. The capillary pressure as a function of the gas saturation in the small-pore medium is involved in the Darcy velocity.

According to the invention, with a flow from the large pores to the fracture via the small pores and, what is more, by imposing a capillary pressure only in the small pores, the gas capillary blockage phenomenon is taken into account,

d) the fluid exchanges occur exclusively between the fracture media between two adjacent cells of the geological reservoir. In other words, there is no fluid exchange between the small-pore and/or large-pore media of a first cell and the small-pore and/or large-pore media of a second cell that would be connected to the first cell. In yet other words, the exchanges occur from one cell to the next only via the fractures.

According to the invention, the fluid flows in the geological reservoir studied are simulated by use of the simulator according to the invention as described above, of the gridded representation of this geological reservoir and of the porosity models determined, calibrated and assigned in each cell of this gridded representation.

Fluid production curves such as gas recovery factor curves, oil recovery curves, a gas/oil ratio are for example obtained at the end of this step.

According to an implementation of the invention, a nested ring type discretization is further used for the simulator according to the invention, for the two media representative of the porous matrix, that is for the small-pore medium and the large-pore medium. Advantageously, the discretization according to the model known as “MINC model” (Multiple Interacting Continua), described in the document (Pruess, 1985), is used. More precisely, according to this implementation of the invention, in order to account for the very slow transient regime between matrix and fracture and the internal exchanges between small pores and large pores, the small-pore and large-pore matrix media are subdivided into MINC cells. The flow within the large pores and within the small pores is considered to be parallel; the matrix permeability thus is the sum of the permeabilities of the small-pore and large-pore media. The flow between the small-pore medium and the large-pore medium is similar to that of a double-medium model between the fracture cell and the first external MINC cell of the small-pore medium. The internal flows in the large pores and the small pores follow a MINC model. According to an implementation of the invention, the flow between the large-pore medium and the small-pore medium follows a particular MINC model, where the exchange surface between large pores and small pores is the ring in the middle of two matrix MINC cells and the exchange distance as the size of a matrix MINC cell. The transmissibility between the large-pore medium and the small-pore medium is proportional to this surface and inversely proportional to this distance. According to this implementation of the invention, one of the flow parameters of the porosity model can be a parameter α representative of the proportionality of the transmissibility from the large-pore medium to the small-pore medium.

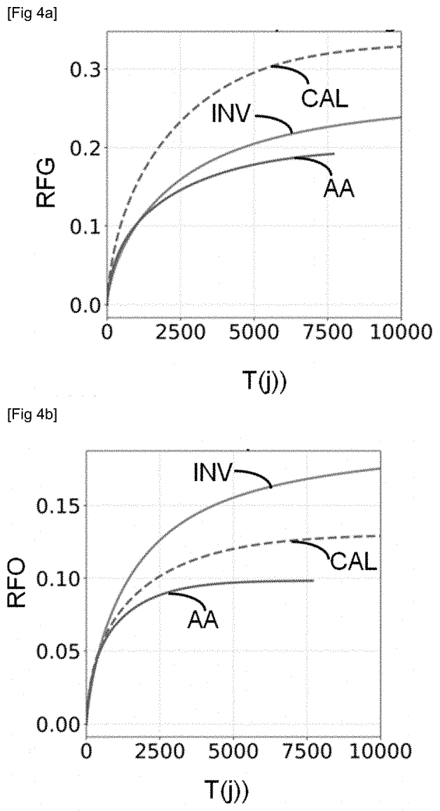

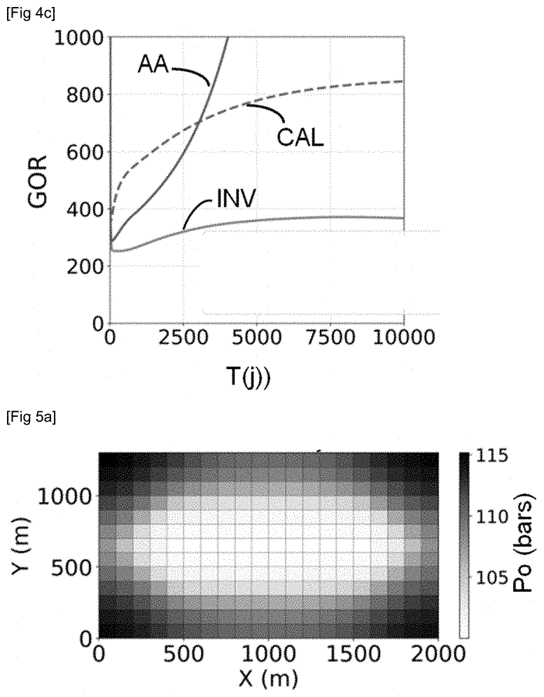

By way of illustration, FIGS. 4a, 4b and 4c show production curves (gas recovery factor (RFG), oil recovery factor (RFO) and gas-oil ratio (GOR) as a function of time T in days) obtained with the method according to the invention (INV) applied to the geological reservoir having the pore size distributions as shown in FIG. 1b. The production trend relative to the unconfined fluid follows the observations made with a fine grid and in-situ observation (CAL). Confinement increases the production of oil and decreases the production of gas and the GOR. By way of comparison, these curves obtained with a double-porosity model of the prior art (AA), confined with a radius size corresponding to the calibration parameter, have been plotted. This model, commonly used in the literature, has a behaviour relative to the unconfined fluid that is not at all in accordance with the observations.

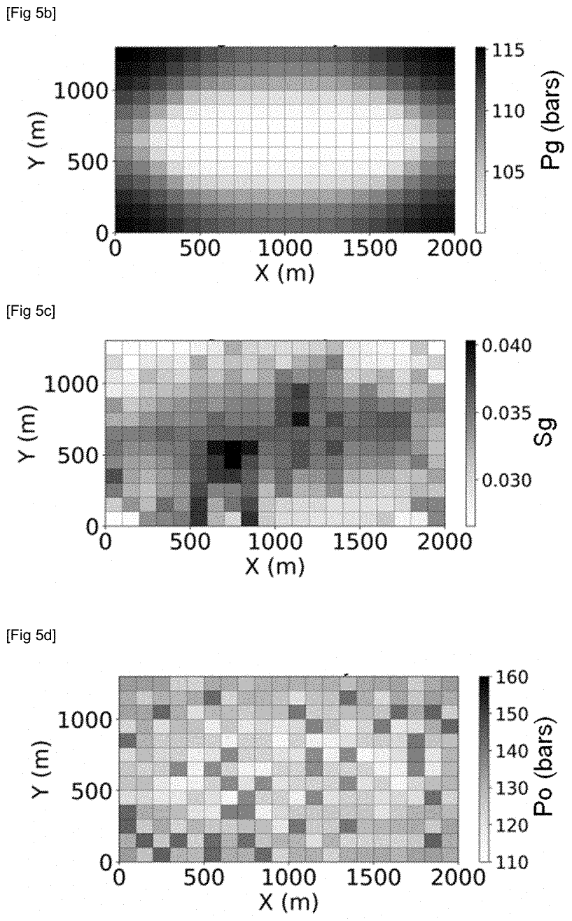

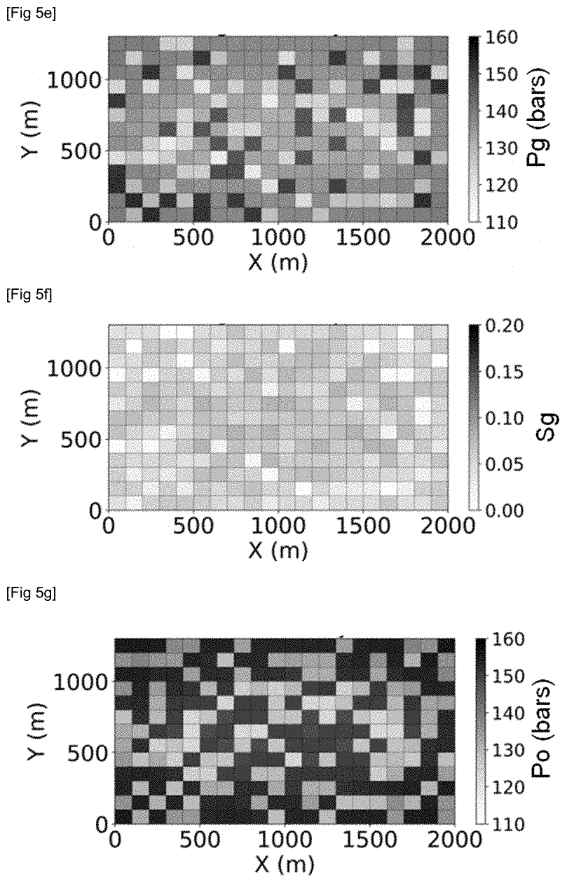

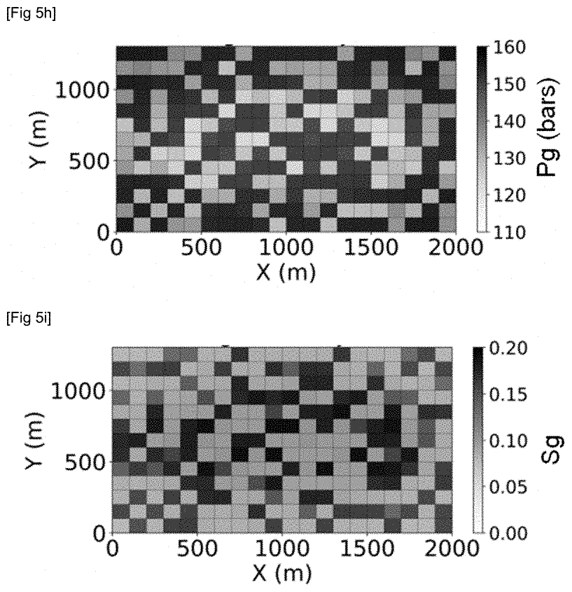

Furthermore, FIGS. 5a to 5i illustrate, for a given time and at any point of a section at constant z in the geological reservoir being studied, oil pressure fields (Po), gas pressure fields (Pg), gas saturations (Sg) for each of the media of the triple-porosity model (FIGS. 5a to 5c for the fracture medium, FIGS. 5d to 5f for the small-pore medium and FIGS. 5g to 5i for the large-pore medium). It can be noted in these figures that the gas saturation is higher in the large pores than in the small pores because the bubble point is decreased in the small pores and because of the capillary blockage exerted in the small pores whose oil/gas capillary pressure is not zero. Indeed, the oil/gas pressure is similar in the large pores and it is different in the small pores.

Thus, according to the first aspect thereof, the present invention enables more precise simulation of the flows of a fluid in a fractured geological reservoir with a heterogeneous pore size. These advantages are obtained by use of a flow simulator implementing a triple-porosity model dividing the matrix medium into a large-pore medium and a small-pore medium, and allowing accounting for the very slow transient regime between porous matrix and fractures, notably via a thermodynamics depending on the pore size and by taking into account of capillary pressure heterogeneities.

It is clear that the method according to the invention comprises steps carried out by use of an equipment (a computer workstation for example) including data processing (processor) and data storage means (a memory, in particular a hard drive), as well as an input and output interface for data input and method results output.

In particular, the data processing are configured for implementing the simulation of the flows within the geological reservoir being studied, by use of a flow simulator according to the invention as described above.

Besides, the invention relates to a computer program product downloadable from a communication network and/or recorded on a computer-readable medium and/or processor executable, comprising program code instructions for implementing the method as described above, when said program is executed on a computer.

5. Exploitation of the Fluid Contained in the Geological Reservoir

This step is carried out within the context of the invention according to a second aspect of the invention, which relates to a method for exploiting a fluid contained in the geological reservoir being studied. The fluid of interest contains hydrocarbons in form of an oil phase and a gas phase.

This step determines at least one exploitation scheme for the hydrocarbons contained in the geological reservoir being studied. In general, an exploitation scheme comprises a number, a geometry and a site (position and spacing) for the injection and production wells to be drilled through the reservoir being studied and to be equipped. An exploitation scheme can also comprise a type of enhanced recovery of the hydrocarbons contained in the reservoir, such as recovery through injection of a solution containing one or more polymers, CO₂ foam, etc. An optimum hydrocarbon reservoir exploitation scheme must for example allow having a high recovery rate for the hydrocarbons trapped in the geological reservoir, over a long exploitation time, and requiring a limited number of wells. In other words, a specialist predefines evaluation criteria according to which a geological reservoir fluid exploitation scheme is considered to be efficient enough to be implemented for the geological reservoir studied.

According to the invention, the exploitation scheme for the hydrocarbons of the geological reservoir being studied is determined by use of the flow simulator according to the invention, by implementing notably the triple-porosity model as described in step 2 above, as well as a thermodynamic flash as described in step 4 above, implementing flows as described in step 4 between the media of the triple-porosity model. Such a reservoir simulator can be established from the flow simulator Puma Flow® (IFP Energies nouvelles, France), which is modified to include the characteristics as described in step 4 above.

In general, at any time t of the simulation, the flow simulator solves all of the flow equations specific to each cell and it delivers values solution to the unknowns (saturations, pressures, concentrations, temperature, etc.) predicted at this time t. This solution provides knowledge of the amounts of oil produced and of the state of the reservoir (pressure distribution, saturations, etc.) at the time being considered. By use of the porosity models that are determined, calibrated and assigned in each cell of the gridded representation as described in the steps above, the flow simulator according to the invention reliably allows prediction notably of the oil and gas productions for a given exploitation scheme.

According to an implementation of the invention, various exploitation schemes are defined for the fluid contained in the geological reservoir being studied and at least one criterion, such as the amount of hydrocarbons produced with each of the various exploitation schemes, the curve representative of the production evolution over time in each well, the gas-oil ratio (GOR), etc., is estimated using the flow simulator according to the invention. The scheme according to which the hydrocarbons contained in the reservoir are really exploited can then correspond to the one meeting at least one of the evaluation criteria of the various exploitation schemes. According to the invention, flow simulations are performed for injection-production well sites using the simulator according to the invention and the porosity models are determined, calibrated and assigned in each cell of the gridded representation, then the exploitation scheme according to which the fluid of the geological reservoir is to be exploited for each well site is determined and the site meeting at least one of the predetermined evaluation criteria is selected. Advantageously, flow simulations can also be carried out for enhanced recovery types, by use of the simulator according to the invention and of the porosity models are determined, calibrated and assigned in each cell of the gridded representation, then the exploitation scheme according to which the fluid of the geological reservoir is to be exploited for each enhanced recovery type is determined and the enhanced recovery meeting at least one of the predetermined evaluation criteria is selected.

Then, once the exploitation scheme determined, the hydrocarbons trapped in the oil reservoir are exploited according to this exploitation scheme, notably at least by drilling the injection and production wells of the exploitation scheme that is thus determined, to produce the hydrocarbons, and by setting up the production infrastructures required for developing this reservoir. In cases where the exploitation scheme was further determined by estimating the reservoir production associated with different enhanced recovery types, the type(s) of additives (polymer, surfactant, CO₂ foam) selected as described above are injected into the injection well.

The exploitation scheme can of course keep developing over the duration of an exploitation of hydrocarbons of a geological reservoir, depending on the reservoir knowledge acquired during exploitation, the improvements in the various technical fields involved in the exploitation of a hydrocarbon reservoir (improvements in the field of drilling, enhanced recovery for example).

1-8. (canceled)

9. A computer-implemented method for simulating fluid flows in a fractured geological reservoir having a heterogeneous pore size, the fluid comprising an oil phase and a gas phase, wherein a first gridded representation of the reservoir is constructed from the measurement of properties relative to the reservoir, comprising:
A. determining from measurements performed in the laboratory on a plurality of samples from various spatial positions in the geological reservoir, pore size distribution classes and assigning a pore size distribution class to each of the cells of the first gridded representation;
B. constructing for each of the pore size distribution classes, a porosity model representative of the pore size distribution, the porosity model comprising a first medium representative of the pores of the distribution whose dimension is in a first range, a second medium representative of the pores of the distribution whose dimension is in a second range and a third medium representative of the fractures of the distribution, the dimensions of the pores of the first range being greater than the dimensions of the pores of the second range, the porosity model being further described by flow parameters for each of the media, the flow parameters comprising at least one equivalent dimension of the pores of the second medium and a capillary pressure depending on a saturation with the gas phase of the fluid in the second medium;
C. calibrating for each of the pore size distribution classes, at least part of the parameters of the porosity model representative of the pore size distribution and assigning the calibrated porosity model in each of the cells of the first gridded representation to which the class is assigned;
the flows of the fluid in the geological reservoir are simulated by use of the first gridded representation and by a first flow simulator, the first simulator implementing at least:
the calibrated porosity models assigned in each of the cells of the first gridded representation;
for the second medium, a thermodynamic equation of state accounting for the equivalent dimension of the pores of the second medium;
within the porosity models, exchanges of the fluid occurring between the first medium and the second medium and between the second medium and the third medium of a single porosity model, accounting for capillary pressure depending on the saturation with the gas phase of the fluid in the second medium; and
between at least two of the porosity models, exchanges of the fluid exclusively occur between the third media of the two porosity models.

10. A method as claimed in claim 9, wherein the calibration for one of the pore size distribution classes is performed with the following steps:
a) simulating the flows by use of the first simulator and of the porosity model representative of the pore size distribution class, the porosity model being defined by the flow parameters of the model, and obtaining production curves depending on the flow parameters of the porosity model;
b) measuring differences between the production curves depending on the flow parameters of the porosity model and predetermined reference production curves, and correcting at least part of the flow parameters of the porosity model to minimize the differences,
c) repeating steps a) and b) until the differences are below a predetermined threshold, the flow parameters of the porosity model for repeating of the steps a) and b) corresponding to the corrected parameters in a previous repeating of the repeating.

11. A method as claimed in claim 10, wherein the reference curves are determined with steps of:
i) constructing a second gridded representation representative of the pore size heterogeneity of the pore size distribution class with dimensions of the cells of the second gridded representation being determined to account for effects induced by the pore size of the distribution; and
ii) simulating flows by use of the second gridded representation and of a second flow simulator implementing at least one thermodynamic equation of state taking account for the dimensions of the pores of the pore size distribution class, and obtaining production curves of the fluid relative to the second gridded representation.

12. A method as claimed in claim 10, wherein the part of the corrected flow parameters comprises the equivalent dimension of the pores of the second medium, an equivalent permeability and porosity of the second medium or an equivalent permeability and porosity of the first medium, and a parameter representative of proportionality of transmissibility from the first medium to the second medium.

13. A method as claimed in claim 11, wherein the part of the corrected flow parameters comprises the equivalent dimension of the pores of the second medium, an equivalent permeability and porosity of the second medium or an equivalent permeability and porosity of the first medium, and a parameter representative of proportionality of transmissibility from the first medium to the second medium.

14. A method as claimed in claim 9, wherein the laboratory measurements are performed by use of a mercury porosimetry method and a nitrogen adsorption/desorption method.

15. A method as claimed in claim 10, wherein the laboratory measurements are performed by use of a mercury porosimetry method and a nitrogen adsorption/desorption method.

16. A method as claimed in claim 11, wherein the laboratory measurements are performed by use of a mercury porosimetry method and a nitrogen adsorption/desorption method.

17. A method as claimed in claim 12, wherein the laboratory measurements are performed by use of a mercury porosimetry method and a nitrogen adsorption/desorption method.

18. A method as claimed in claim 9, wherein the first medium and second medium are discretized using a nested ring type discretization.

19. A method as claimed in claim 10, wherein the first medium and second medium are discretized using a nested ring type discretization.

20. A method as claimed in claim 11, wherein the first medium and second medium are discretized using a nested ring type discretization.

21. A method as claimed in claim 12, wherein the first medium and second medium are discretized using a nested ring type discretization.

22. A method as claimed in claim 13, wherein the first medium and second medium are discretized using a nested ring type discretization.

23. A method as claimed in claim 14, wherein the first medium and second medium are discretized using a nested ring type discretization.

24. A tangible computer program product comprising program code instructions executed on a computer which implements the method as claimed in claim 9.

25. A method for exploiting a fluid contained in a fractured geological reservoir having a heterogeneous pore size distribution, which performs the method as claimed in claim 9, wherein, from at least the simulation of the flows in the geological reservoir, an exploitation scheme for the geological reservoir comprising at least one site for at least one of an injection well and at least one of production well is determined, and exploiting fluid of the geological reservoir by drilling the wells of the site and by providing the drilled well with exploitation infrastructures.
